(19) United States
(12) Patent Application Publication
(10) Pub. No.: US 2005/0131240 A1
(43) Pub. Date: Jun. 16, 2005

(54) Process for separating tocopherol homologues

(76) Inventor: Anil B. Khare, Crystal, MN (US)

Correspondence Address: Edna Vassilovski, Esq.;Cargill, Incorporated, Legal Department, P.O. Box 5624, Minneapolis, MN, 55440-5624, US

(21) Appl. No.: 10/982,435

(22) Filed: Nov. 4, 2004

Related U.S. Application Data
(60) Provisional application No. 60/517,332, filed on Nov. 4, 2003.

(51) Int. Cl. C07D 311/04

(52) U.S. Cl. 549/413

(57) ABSTRACT

Methods for separating gamma and delta homologues of tocopherol are described.

### CROSS-REFERENCE TO RELATED APPLICATION

This application claims the benefit of U.S. Provisional Patent Application 60/517,332 filed Nov. 4, 2003, the entire disclosure of which is incorporated herein by reference.

### TECHNICAL FIELD

This invention relates to a process for separating tocopherol homologues, and more particularly, to separating gamma and delta tocopherols using an affinity resin such as an ion exchange resin.

### BACKGROUND

Tocopherols are widely used for their antioxidant and Vitamin E activity. There are four main tocopherol homologues found in vegetable oils: alpha, beta, gamma, and delta-tocopherol. Chemically, all four are methyl derivatives of tocol[2-methyl-2-(4′,8′,12′-trimethyltridecyl)-6-chromanol]. Alpha-tocopherol, with its completely methylated ring and saturated side chain, possesses the highest biological activity. Alpha-tocopherol and its acetate are the forms most used commercially. The naturally occurring d form of alpha tocopherol is the most active isomer physiologically with the racemic synthetic dl-alpha-tocopherol and its esters being less potent on a weight for weight basis than the d form. Alpha-tocopherol acetate is the principal commercial form of vitamin E in medicine. Tocopherols, including gamma and delta tocopherols, have been used in foods as antioxidants to retard rancidity in fatty materials. Tocopherols have also been used in skin creams and lotions. Because of their antioxidant activity, these compounds also may have anticancer activity.

Tocopherols are found in many foods in an unesterified form. The highest concentrations are found in the cereal grain oils. Crude corn and wheat oils for example may contain 200 mg of tocopherol per 100 g of oil. Certain vegetable oils, such as coconut oil, however, are practically devoid of tocopherols. Similarly, the proportion of the various isomers also varies widely. For example, about 90% of the tocopherol in safflower oil is alpha-tocopherol, whereas only about 20% of corn and soybean oil is in the alpha form. The gamma form predominates in corn oil, while the gamma and delta forms predominate in soybean oil. Wheat oils contain mixtures of tocopherol, with up to 65% in the beta form.

### SUMMARY

The invention includes a method for separating gamma and delta tocopherols. As described herein, methods of the invention allow gamma and delta tocopherols to be separated and obtained in a purified form while minimizing the required amount of solvent and holding time on a column.

Accordingly, in one aspect the invention provides a method of separating gamma and delta tocopherols. The method includes a) loading an ion exchange resin with a tocopherol composition to form a loaded resin, where the tocopherol composition consists essentially of gamma and delta tocopherols. The method also includes b) selectively eluting gamma tocopherols from the loaded resin with an organic solvent and c) selectively eluting delta tocopherols from the loaded resin with an acidified, organic solvent. Step c) can be performed subsequently to step b). The organic solvent in step b) can be methanol, which may contain an acid, such as an organic acid. The acid used in step b) can be in an amount of from 0.1% to 3% by weight, or any value therebetween. The acidified organic solvent of step c) can be methanol containing about 3% to 5% of an acid, such as an organic acid (e.g., acetic acid). Selective elution of gamma or delta tocopherols can result in a gamma or delta tocopherol fraction, respectively, having a purity of from about 85% to about 100%, e.g., 85% to 95%, 90% to 95%, 95% to 99%, 98% to 100%, or any value therebetween.

The ion exchange resin can be contained in a column. The ion exchange resin can be a porous, OH-anion exchange resin. In some embodiments, the method can include regenerating the ion exchange resin, e.g., using sodium hydroxide.

The ratio of the load of tocopherols to the resin volume can range from 115 to 350 g/L, or from 145 to 300 g/L. The tocopherol- composition can be a soybean distillate. The tocopherol composition can contain 65 to 75% gamma tocopherols and 15 to 25% delta tocopherols.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description.

### DETAILED DESCRIPTION

The invention provides a method for separating different tocopherol homologues from a composition consisting essentially of gamma and delta tocopherols. Such a composition can be produced from soybean oil. For example, crude soybean oil can be distilled and the resulting distillate can include phytosterols, sterol esters, free fatty acids, mono-, di-, and tri-glycerides, squalene, and tocopherols. The distillate can be transesterified to isolate a mixture containing tocopherols and sterols. The sterols can be crystallized and removed (e.g., by filtration) to produce a composition containing primarily gamma and delta tocopherols. In particular, such a composition can include 60 to 70% gamma tocopherol and 15 to 25% delta tocopherol, with less than 8% alpha and beta tocopherols. In certain embodiments, the sterols and alpha tocopherols can be removed by use of an ion exchange column, such as Purolite™ A503, or Rohm and Hass Ambrelyst™ A-26 OH-type. In such cases, such a composition can include 60 to 70% gamma tocopherol and 15 to 25% delta tocopherol, with less than 1% alpha tocopherols.

Tocopherol homologues can be separated from one another using an affinity resin such as an ion exchange resin. The affinity resin may be in the form of a slurry or contained within a column. Suitable ion exchange resins are porous (e.g., cross-linked from about 1-8%) and basic. For example, the ion exchange resin can be a DOWEX ion exchange resin from Dow Chemical Co., an Amberlite™ resin from Rohm and Haas, a DIAION® resin from Mitsubishi Chemical, a Purolite A-500™ hydroxide anion exchange resin from Rohm and Hass, or the Mitsubishi HPA-25™ or MAO1SS ™ resins. In particular, the Dowex-1X2 ion exchange resin (50-100 mesh) from Dow Chemical can be used. Typically, such resins are sold in the chloride form and can be converted to the hydroxide (OH) form using an alkaline solution (e.g., an aqueous solution of sodium or potassium hydroxide). After converting the resin to the OH form, the resin can be rinsed with water until the pH reaches neutrality. Typically, 5-8 column volumes of water are sufficient to reach neutrality. The mobile phase of the resin can be prepared for loading by rinsing the resin in an organic solvent, typically the organic solvent to be used for loading and elution.

A tocopherol composition can be loaded onto an ion exchange resin using an organic solvent (e.g., a polar solvent such as methanol). The ratio of the load of tocopherols to the resin volume can range from 80 to 500 g/L (e.g., 100 to 400 g/L, 115 to 350 g/L, or 145 to 300 g/L). Gamma tocopherols can be selectively eluted relative to delta tocopherols using an organic solvent such as methanol. Any alpha tocopherol in the tocopherol composition also is generally eluted with this solvent. Delta tocopherols can be selectively eluted from the resin by lowering the pH of the eluting solvent with an acid (e.g., an organic acid). For example, an acidified polar solvent (e.g., methanol with an organic acid such as 3 to 5% acetic acid) can be used. Elution of the gamma and delta tocopherols can be monitored using, for example, UV absorbance at 256 nm and thin layer chromatography (TLC). Depending on the elution profile and time desired, the elution steps described herein can be gradient or isocratic.

Selective elution of gamma tocopherols relative to delta tocopherols can result in a gamma tocopherol fraction having a purity of from about 85% to about 100%, e.g., 85% to 95%, 90% to 95%, 95% to 99%, 98% to 100%, or any value therebetween. Similarly, selective elution of delta tocopherols from the resin can result in a delta tocopherol fraction having a purity of from about 85% to about 100%, e.g., 85% to 95%, 90% to 95%, 95% to 99%, 98% to 100%, or any value therebetween.

Acidifying the organic solvent used to elute the gamma tocopherols can decrease the elution time of the gamma tocopherols. The acid used to acidify can be an organic acid, including the organic acid used to elute the delta tocopherols, such as acetic acid. The organic solvent can be acidified to a degree effective to elute the gamma tocopherols in a desired elution time frame, e.g., an elution time that is smaller than the comparable elution time of the gamma tocopherols in the absence of the acid. The amount of acid added to the organic solvent can vary depending on a number of factors, including the elution time desired and/or the purity profile needed for the eluted gamma fraction. An organic solvent can be acidified with an acid in a range of 0.1% to 3% by weight (e.g., 0.2 to 2.8%, 0.5 to 2.5%, or 0.8 to 1.5%), or any particular value therebetween. Typically, the acid is added in an amount from 0.1% to 2%. Additionally, increasing the acid concentration of the organic solvent will increase the co-elution of delta tocopherols in the gamma fraction, especially in the range from 2% to 3% acid concentration and beyond.

After the delta tocopherols are eluted, the ion exchange resin can be regenerated by methods known to those of ordinary skill in the art, such as using, for example, a solution of sodium hydroxide (e.g., 2-5 bed volumes of 2-5% NaOH solution). Regenerated resins can be re-used.

Systems containing a variety of elements (e.g., valves, pumps, or fraction collectors) and columns can be configured to automate the separation of the tocopherol homologues. Typically, the tocopherols are separated at room temperature (about 25° C.), although the temperature can be elevated to about 60° C. to reduce the holding time on the resin. Flow rates suitable for separating tocopherol homologues can vary depending on column size and pump used. For example, flow rates can range from about 40 mL/minute to about 400 mL/minute. In some embodiments, the flow rate can be about 225 to 275 mL/minute (e.g., 250 mL/minute). Increasing the flow rate can reduce the amount of solvent required.

The invention will be further described in the following examples, which does not limit the scope of the invention described.

### EXAMPLES

### Example 1

### Large Scale Separation of Gamma and Delta Tocopherols from Soybean Distillate

Gamma and delta tocopherols were separated from a starting tocopherol mix containing 68.3% gamma tocopherol, 5.5% alpha tocopherol, 1.3% beta tocopherol, and 19.9% delta tocopherol. A 33.2L³ stainless steel column (182 cm×15.25 cm) containing 28.3 L (˜19.65 kg) of Dowex- 1X2 ion-exchange resin (50-100 mesh) was washed with 60 L of a 4% sodium hydroxide solution to convert the resin to the OH form. The column was rinsed with de-ionized water until the pH reached neutrality.

The mobile phase of the column was prepared for sample loading by rinsing the column with 75 L of methanol. The column was loaded with 4050 g of the tocopherol mix in methanol (20 L total volume). The flow rate for loading and elution was 250 mL/min. Elution was monitored by UV absorbance at 256 nm and TLC. Gamma tocopherol was eluted using methanol (total of 709 L of methanol, 47.5 hours). Delta tocopherol was eluted using 75 L of 4% acetic acid/methanol solution.

### Example 2

### Separation of Gamma and Delta Tocopherols from Soybean Distillate

A glass column (5 cm×118 cm) was packed with Dowex 1 -X2 ion-exchange resin (200 mesh). The resin was converted to the OH form by washing with 5 L of 4% sodium hydroxide/water solution. The column was rinsed with deionized water until the pH of the eluate reached neutrality (˜50 L water, ˜pH 7.0) then 4 L of methanol to prepare the mobile phase. The column was loaded with 300 g of the mixed tocopherol sample, diluted 1:1 in methanol. Total tocopherol concentration was 5.6% alpha, 71.0% gamma, 1.3% beta, and 20.4% delta. The flow rate was approximately 150 mL/min. Fraction separation was determined by absorbance at 256 nm and TLC of eluted fractions. The experiment was conducted at room temperature and methanol was used as the mobile phase. Peak absorbencies showed separation of impurities, alpha, and gamma tocopherols. When the detector showed that most of the gamma had eluted, the solvent was changed to a 5% acetic acid/methanol solution, which eluted the delta tocopherols.

Table 1 summarize experiments with different column loads and solvent ratios.

TABLE 1

| | | | Fraction | | | Purity |
|---|---|---|---|---|---|---|
| Sample | Fraction | Load | Load | Mass | Mass/Load | (Area) |
| 79-1 | Gamma | 50 | 36.15 | 22.85 | 63.2 | 92.86 |
| 79-2 | Delta | 50 | 10.2 | 9.75 | 95.6 | 96.63 |
| 79-3 | Gamma | 50 | 36.15 | 8.83 | 24.4 | 93.66 |
| 84-3 | Alpha | 280 | 15.68 | 15.4 | 98.2 | N/A |
| 84-4 | Gamma | 280 | 202.44 | 131 | 64.7 | N/A |
| 84-5 | Gamma | 280 | 202.44 | 110.94 | 54.8 | N/A |
| 84-6 | Delta | 280 | 57.12 | 37.86 | 66.3 | N/A |
| 86-1 | Alpha | 300 | 15.8 | 12.84 | 81.3 | 94.71 |
| 86-2 | Gamma | 300 | 216.9 | 89.77 | 41.4 | 93.33 |
| 86-3 | Gamma | 300 | 216.9 | 118.65 | 54.7 | 97.33 |
| 86-4 | Delta | 300 | 61.2 | 73.8 | 120.6 | 98.63 |
| 88-1 | Alpha | 300 | 15.8 | 13.65 | 86.4 | 63.24 |
| 88-2 | Gamma | 300 | 216.9 | 147.26 | 67.9 | 90.22 |
| 88-3 | Gamma | 300 | 216.9 | 69.54 | 32.1 | 94.59 |
| 88-4 | Delta | 300 | 61.2 | 59.73 | 97.6 | 98.82 |
| 90-1 | Alpha | 400 | 22.4 | 16.89 | 75.4 | N/A |
| 90-2 | Gamma | 400 | 289.2 | 72.46 | 25.1 | N/A |
| 90-3 | Gamma | 400 | 289.2 | 78.52 | 27.2 | N/A |
| 90-4 | Gamma | 400 | 289.2 | 91.69 | 31.7 | N/A |
| 90-5 | Gamma | 400 | 289.2 | 28.74 | 9.9 | N/A |
| 90-6 | Delta | 400 | 289.2 | 107.89 | 132.2 | N/A |

Table 2 summarizes results from different resins and solvents.

TABLE 2

| | Resin | Toco | Alpha/Gamma | Elution | | | |
|---|---|---|---|---|---|---|---|
| | Vol. | load | elution volume | Solvent:Toco | Toco:resin | | |
| Resin | (mL) | (g) | (L) | ratio (L per g) | ratio (g/L) | Success? | Comments |
| DOWEX-1X2 | 280 | 42 | 5.25 | 0.125 | 150 | + | |
| DOWEX-1X2 | 280 | 50 | 8.10 | 0.162 | 179 | + | |
| DOWEX- | 2100 | 330 | 42.00 | 0.127 | 157 | − | |
| MAOISS | | | | | | | |
| DOWEX-1X2 | 2400 | 280 | 14.00 | 0.050 | 117 | − | |
| DOWEX-1X2 | 2400 | 300 | 31.00 | 0.103 | 125 | + | |
| DOWEX-1X2 | 2400 | 300 | 32.70 | 0.109 | 125 | + | Slower |
| | | | | | | | elution |
| | | | | | | | speed |
| DOWEX-1X2 | 2400 | 400 | 60.40 | 0.151 | 167 | + | |
| DOWEX-1X2 | 280 | 42 | .845 | 0.020 | 150 | − | Ethanol |
| MAO 1SS | 280 | 42 | 2.50 | 0.060 | 150 | − | |
| DOWEX-1X2 | 280 | 42 | 10.00 | 0.238 | 150 | + | KOH wash |
| Purolite A- | 280 | 40 | 1.80 | 0.045 | 143 | − | |
| 500OH | | | | | | | |
| HPA25 | 280 | 40 | 1.05 | 0.026 | 143 | − | |
| DOWEX-1X2 | 100 | 30 | 5.10 | 0.170 | 300 | + | Slurry |
| DOWEX-1X2 | 28000 | 4050 | 708 | 0.175 | 145 | + | Pilot |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for separating gamma and delta tocopherols, said method comprising:
a) loading an affinity resin with a tocopherol composition to form a loaded resin, said tocopherol composition consisting essentially of gamma and delta tocopherols;
b) selectively eluting gamma tocopherols from said loaded resin with an organic solvent; and
c) selectively eluting delta tocopherols from said loaded resin with an acidified organic solvent.

2. The method of claim 1, wherein said organic solvent of step b) is methanol.

3. The method of claim 1, wherein said organic solvent of step b) is methanol containing an acid.

4. The method of claim 3, wherein said acid is an organic acid.

5. The method of claim 1, wherein said acidified organic solvent of step c) is methanol containing an acid.

6. The method of claim 1, wherein said acid is an organic acid.

7. The method of claim 4, wherein said organic acid concentration is between 0.1% and 2% acetic acid by weight.

8. The method of claim 6, wherein said organic acid is between 3% and 5% acetic acid by weight.

9. The method of claim 1, wherein the ratio of the load of tocopherols to the resin volume ranges from 115 to 350 g/L.

10. The method of claim 9, wherein the ratio of the load of tocopherols to the resin volume ranges from 145 to 300 g/L.

11. The method of claim 1, wherein said ion exchange resin is contained in a column.

12. The method of claim 1, wherein said tocopherol composition contains 65% to 75% gamma tocopherols and 15% to 25% delta tocopherols.

13. The method of claim 1, wherein said tocopherol composition is a soybean distillate.

14. The method of claim 1, wherein said ion exchange resin is an ion exchange resin.

15. The method of claim 1, wherein said ion exchange resin is a porous, OH-anion exchange resin.

16. The method of claim 4, wherein said organic acid in step b) comprises an organic acid in an amount effective to decrease the elution time of said gamma tocopherols relative to the elution time of said gamma tocopherols in the absence of said organic acid.

17. The method of claim 1, wherein step c) is performed after step b).
